(19) United States
(12) Patent Application Publication
(10) Pub. No.: US 2020/0114041 A1
(43) Pub. Date: Apr. 16, 2020

(54) SYSTEMS AND METHODS FOR DELIVERING A POLYMERIC MATERIAL TO A TREATMENT SITE DURING A RADIO FREQUENCY ABLATION PROCEDURE

(71) Applicant: Avent, Inc., Alpharetta, GA (US)

(72) Inventors: Guillermo Alas, Alpharetta, GA (US); Michael G. Smith, Alpharetta, GA (US); Sherry Adesina, Tucker, GA (US); Alencia Washington, Roswell, GA (US); Mark Lavigne, Alpharetta, GA (US)

(21) Appl. No.: 16/653,135

(22) Filed: Oct. 15, 2019

Related U.S. Application Data
(60) Provisional application No. 62/745,652, filed on Oct. 15, 2018.

(51) Int. Cl. A61L 24/00; A61B 18/14; A61L 24/04

(52) U.S. Cl. CPC A61L 24/001; A61B 2018/00029; A61L 24/046; A61B 18/1477

(57) ABSTRACT

An example temperature-controlled system is described herein. The system can be used for delivering a thermoresponsive polymer to a treatment site, for example, during an RF ablation procedure. The system can include a syringe capable of maintaining the thermoresponsive polymer liquid at a temperature below its liquid-solid phase transition temperature.

### CROSS-REFERENCE TO RELATED APPLICATIONS

This application claims priority to U.S. Provisional Application No. 62/745,652, filed Oct. 15, 2018, which is herein incorporated by referenced in its entirety.

### TECHNICAL FIELD

The present disclosure relates to a temperature-controlled system for delivering a stimuli-responsive biomaterial to a treatment site.

### BACKGROUND

Pain in the knees, hip, or back may manifest itself to such a high degree that the patient's quality of life is greatly diminished. In such cases, a physician may perform an electrosurgical procedure applying high frequency (e.g., radiofrequency (RF) energy to treat, cut, ablate or coagulate tissue structures including neural tissue. In this minimally invasive procedure, a probe is inserted into a patient's body and placed at the nerve site responsible for the pain. High frequency energy is delivered to the region of tissue from an energy source such as a generator via an active electrode of the probe. The resistance of tissue, located proximate the active electrode of the probe, to the high frequency energy causes the tissue temperature to rise and a lesion is formed at the nerve site. While this desyncing of the nerve provides the desired pain relief, often, the tissue proximate to the probe heats up faster than tissue farther away from the probe limiting the size of the lesion.

Following a radiofrequency ablation (RFA) procedure, angiogenesis will begin at the ablation site. This is a typical response for nerve tissue that has undergone catastrophic damage. However, due to the destructive nature of RFA therapies, the nerve is unable to sync with its pre-ablation downstream counterpart. As a result, the distal/downstream nerve site will degrade from a process known as Wallerian degradation. Wallerian degradation prevents the proper re-growth between the two sites and results in the nerve structure at the ablation site growing in an uncontrolled manner known as a growth cone. It is hypothesized that this process is responsible for the pain felt by patients several months to years after the RFA procedure. Angiogenesis can be manipulated by stopping or slowing the regrowth of the nerve tissue. This requires administering a chemical, biological, or combination of growth antagonist to the nerve ablation site. However, several drawbacks exist for this approach, including: the logistics needed to place the growth inhibitor molecule(s), regulate the release of the molecule(s), and sustained release of the growth inhibitor molecule(s) at the ablation site. Instead of prohibiting nerve regrowth, the innovative approach described herein comprises mitigating and manipulating nerve growth at the ablation site to prevent the formation of an unregulated growth cone and further delay the return of pain for the recipients of RFA.

As described in co-pending U.S. application Ser. No. ______, titled “Compositions, Systems, Kits and Methods for Neural Ablation” (Attorney Docket No. 10964-006US1) filed concurrently herewith and incorporated herein by reference, biomaterials can be applied proximate/over the lesion and/or nerve ablation site during a RF procedure to manipulate and mitigate nerve growth. The layer of biomaterial can also serve to provide a physical barrier that prevents immune development. These biomaterials can be provided at the treatment site during/following the RF procedure and would benefit from the claimed delivery system. In some examples, the biomaterial comprises a stimuli-responsive biocompatible polymer, e.g., a thermoresponsive polymer, that must be cooled to its liquid phase temperature for delivery through a syringe and/or introducer cannula. As the polymer warms within the body it solidifies to provide a physical barrier between the lesions site and surrounding tissue. Accordingly, a need in the art exists for a cooled syringe that can maintain the temperature of the polymer below its liquid-solid phase transition temperature. In other examples, the biomaterial comprises a crosslinkable biomaterial formed from a first precursor and a second precursor which react in situ to form a polymeric matrix. In some of these embodiments, the first precursor and the second precursor must be separately stored prior to being combined at the treatment site. Accordingly, a need in the art exits for a double barrel delivery system that maintains the first precursor and the second precursor in separate volumes before combining at the treatment site and/or in the delivery cannula.

### SUMMARY

Systems and methods for delivering a biomaterial to a treatment site are described herein. An example temperature-controlled system for delivering a stimuli-responsive biomaterial to a treatment site can include a syringe that maintains a thermoresponsive polymer liquid contained therein at a temperature below its liquid-solid phase transition temperature. The syringe can include a body defining a volume for containing a thermoresponsive polymer liquid, a distal end of the body includes a delivery tip, and a plunger sized and configured to move within the volume of the body to convey the thermoresponsive polymer liquid through the delivery tip. The body of the syringe can include a cooling mechanism for maintaining an interior surface of the body at a temperature below the liquid-solid phase transition temperature of the thermoresponsive polymer liquid. The cooling mechanism can include an insulative material provided around the body of the syringe. The body of the syringe can also be formed from a double wall vacuum insulated material. The cooling mechanism can also include a cooling pad coupled to the exterior of the body of the syringe. The cooling mechanism can also include a chilled water system where a volume of chilled water is flowed around the body of the syringe. The cooling mechanism can also include a thermoelectric cooler.

The temperature-controlled system can also include an elongated introducer cannula including a central channel sized and configured to receive the delivery tip of the syringe. The system can further include a cooled radiofrequency (RF) probe sized for insertion into the central channel of the introducer cannula, the cooled RF probe being configured to circulate water at a temperature that maintains the introducer cannula at a temperature below the liquid-solid phase transition temperature of the thermoresponsive polymer liquid. The introducer cannula can include at least one of an insulative material provided around the introducer cannula, a double wall vacuum insulated material, a cooling pad coupled to the introducer cannula, a thermoelectric cooler, or a chilled water system for flowing a volume of chilled water around the introducer cannula.

The temperature-controlled system can maintain an interior surface of the body of the syringe at a temperature below about body temperature. The interior surface of the body of the syringe can be maintained at a temperature between about 28° C. and about 42° C.

The temperature-controlled system can maintain an interior surface of the body of the syringe at a temperature below about room temperature. The interior surface of the body of the syringe can be maintained at a temperature between about 19° C. and about 25° C.

The temperature-controlled system can maintain the syringe or the thermoresponsive polymer at a temperature below about 20° C.

The temperature-controlled system can maintain the syringe or the thermoresponsive polymer at a temperature at or below about 4° C.

An example system for delivering a biomaterial to a radio frequency ablation treatment site can include a syringe and an elongated introducer cannula. The syringe can include a body defining a volume for containing a biomaterial, a distal end of the body includes a delivery tip, and a plunger sized and configured to move within the volume of the body to convey the biomaterial through the delivery tip. The elongated introducer cannula can include a central channel sized and configured to receive the delivery tip of the syringe, and an engagement feature provided at a proximal end of the introducer cannula for coupling with a corresponding engagement feature provided at a distal end of the syringe. The syringe and introducer cannula can be movable between an unlocked and locked configuration, where in the unlocked configuration the syringe and introducer cannula engagement features are not coupled, and where in the locked configuration the syringe and introducer engagement features are coupled. In the unlocked configuration axial movement of the plunger within the volume of the body can be prohibited, preventing flow of the biomaterial through the delivery tip. In the locked configuration axial movement of the plunger within the volume can be permitted thereby providing flow of the biomaterial through the delivery tip and into the introducer cannula. The system can further include a restraint mechanism coupled to the plunger, wherein the restraint mechanism is configured to restrict axial movement of the plunger within the volume. The engagement feature provided at the proximal end of the introducer cannula can be a female Luer fitting, and the engagement feature provided at the distal end of the syringe can be a male Luer fitting, where rotational movement between the male and female Luer fittings results in secured coupling between the introducer cannula and the syringe. The engagement feature provided at the proximal end of the introducer cannula can include at least one of a recess, a projection, a tapered surface for press fitting with the syringe, a clip, a thread, a bayonet mount, and the engagement feature provided at the distal end of the syringe can include a corresponding at least one of a projection, a recess, a tapered surface for press fitting with the introducer cannula, a clip, a thread, and a bayonet mount, where coupling movement between the engagement features of the introducer cannula and syringe results in a secured coupling between the introducer cannula and the syringe.

The system can further include a tagging system configured to determine the unlocked and locked configurations of the syringe and introducer cannula. The tagging system can include a radiofrequency identification (RFID) integrated circuit, wherein the RFID integrated circuit confirms alignment of a locking portion of the introducer cannula with a corresponding locking portion of the syringe, where when the RFID integrated circuit determines that the locking portions of the introducer cannula and syringe are not in alignment, the introducer cannula and syringe are in the unlocked configuration, and when the RFID integrated circuit determines that the locking portions of the introducer cannula and syringe are in alignment, the introducer cannula and syringe are in the locked configuration.

The delivery tip can include an 18-gauge, 19-gauge, or 20-gauge needle. At least a portion of the delivery tip can be coupled to the proximal end of the introducer cannula and extends at least partially into the central channel of the introducer cannula.

An example system for delivering a crosslinkable biomaterial to a treatment site can include a syringe having a body defining a first volume for containing a first precursor and a separate second volume separate from the first volume for containing a second precursor, a distal end of the body includes a delivery tip. The syringe can also include a plunger sized and configured to move within the first and second volumes of the body to convey the first and second precursors through the delivery tip. The delivery tip can include an elongated needle defining a first bore and a separate second bore, wherein the first bore is in fluid communication with the first volume of the syringe and the second bore is in fluid communication with the second volume of the syringe, such that the first precursor and the second precursor transmitted through the needle combine as they pass through a distal end of the needle.

The system for delivering a crosslinkable biomaterial can also include a barrier separating the first volume from the second volume is opened such that contents of the first volume and the second volume combine within the syringe before passing through the delivery tip. The system for delivering a crosslinkable biomaterial can also include an elongated introducer cannula including a central channel sized and configured to receive the delivery tip therethrough.

An example method of delivering a stimuli-responsive biomaterial to a treatment site can include inserting an introducer cannula into a body of a patient, wherein a distal end of the introducer cannula is located inside the body of the patient and proximate a treatment site and a proximal end of the introducer cannula is located outside the body of the patient. The method can further include inserting a delivery tip of a syringe into the proximal end of the introducer cannula, wherein a stimuli-responsive biomaterial is contained within a body of the syringe. The method can further include maintaining the stimuli-responsive biomaterial at a temperature below its liquid-solid phase transition temperature. The method can further include depressing a plunger of the syringe to advance the stimuli-responsive biomaterial in its liquid state through a delivery tip of the syringe and to the treatment site.

The method can further include cooling an interior surface of the body of the syringe to a temperature below the liquid-solid phase transition temperature of the stimuli-responsive biomaterial. The method can further include cooling the introducer cannula using a cooled radiofrequency (RF) probe during a cooled RF ablation procedure. The method can further include cooling the introducer cannula to a temperature below the liquid-solid phase transition temperature of the stimuli-responsive biomaterial using a cooled radiofrequency (RF) probe during a cooled RF ablation procedure. The method can further include moving the introducer cannula and syringe between an unlocked and locked configuration, wherein in the unlocked configuration axial movement of the plunger of the syringe is prohibited, and wherein in the locked configuration axial movement of the plunger of the syringe is permitted. The stimuli-responsive biomaterial can be a thermoresponsive polymer comprising a poloxamer.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A is a perspective view of a temperature-controlled system for delivering a thermoresponsive polymer to a treatment site according to implementations described herein.

FIG. 1B is a perspective view of an example temperature-controlled system for delivering a thermoresponsive polymer to a treatment site according to implementations described herein.

FIG. 2 is a perspective view of the temperature-controlled system of FIG. 1A including a delivery needle.

FIG. 3 is a perspective view of the temperature-controlled system of FIG. 1A and an example probe assembly.

FIG. 4 is a perspective view of the example probe of FIG. 3.

FIG. 5 is a perspective view of a system for delivering a polymer to a treatment site according to implementations described herein.

FIG. 6A is a perspective view of a system for delivering a polymer according to implementations described herein.

FIG. 6B is a perspective view of a portion of the mixing chamber of FIG. 6A.

FIG. 7 is a top view of an example probe positioned within an intervertebral disc of a patient.

FIG. 8 is a top view of an example system for delivering a polymer within an intervertebral disc of a patient.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. As used herein, the terms “about” or “approximately,” when used in reference to temperature, mean within plus or minus 10 percentage of the referenced temperature. While implementations will be described for controlling the temperature of and injecting a biomaterial during an RF ablation procedure and/or cooled RF ablation procedure, it will become evident to those skilled in the art that the implementations are not limited thereto, but are applicable for biomaterials other than stimuli-responsive biocompatible polymer, e.g., a thermoresponsive polymers, and crosslinkable biomaterials and/or procedures other than RF ablation.

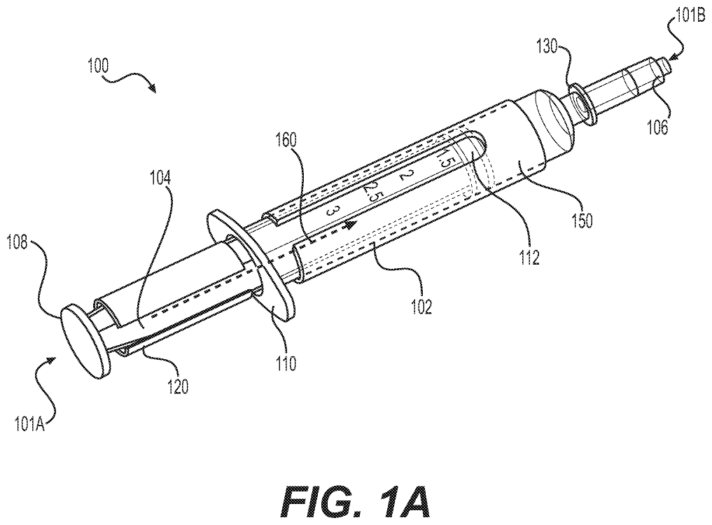

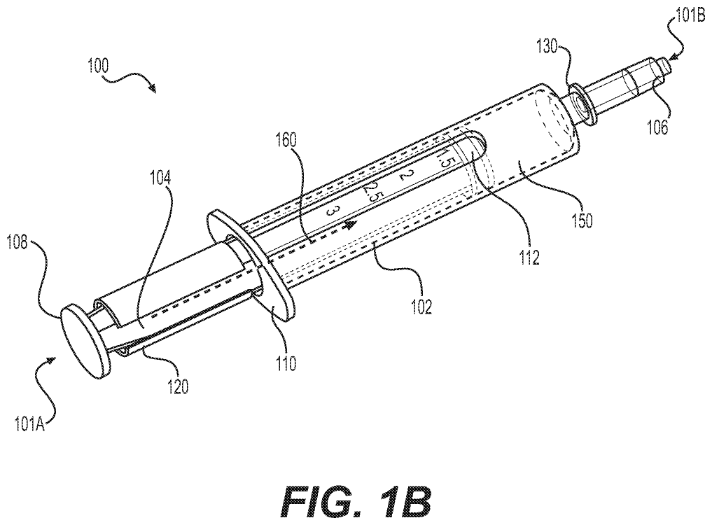

Referring now to FIGS. 1A-8, example temperature-controlled systems are described. The temperature-control system can be used for delivering a stimuli-responsive biocompatible polymer, such as a thermoresponsive polymer, to a treatment site during an electrosurgical procedure, including, for example, an RF ablation procedure. The temperature-control system may be used with systems used for RF treatment of bodily tissue, in particular, cooled probes including some form of temperature and/or impedance monitoring concepts and their corresponding systems, such as those described in U.S. Pat. Nos. 6,896,675, 7,163,536, 7,294,127, 7,824,404, 8,043,287, 8,187,268, 8,361,063, 8,518,036, 8,740,897, 8,864,759, 8,882,755, 8,951,249, 9,364,281, 9,468,275, 10,206,739, 10/327,839, the disclosures of which are incorporated herein by reference.

As used herein, a “thermoresponsive polymer,” “thermosensitive polymer” or “inverse thermosensitive polymer” is a polymer that exhibits a change in physical property in response to temperature. For example, a thermoresponsive polymer can be in a liquid state at a relatively low temperature (e.g., when cooled or refrigerated) but in a gel and/or solid state at a relatively high temperature (e.g., at room temperature or body temperature). In other words, a thermoresponsive polymer can change phase in dependence on temperature. As described in more detail below, the syringe 100 can maintain a thermoresponsive polymer at a temperature below its liquid-solid phase transition temperature such that the thermoresponsive polymer is in a liquid state within the syringe 100 and prior to delivery to the treatment site. A “poloxamer” is an example thermoresponsive polymer. Poloxamers can be used for drug delivery as well as creating a physical barrier between the target nerve and the surrounding tissue. Although poloxamers are described in the examples herein, this disclosure contemplates that the thermoresponsive polymer is not limited to poloxamers.

In some embodiments, the inverse thermosensitive polymer can exhibit a transition temperature of from 10° C. to 37° C. In some cases, the inverse thermosensitive polymer can be a liquid at 4° C., and undergoes a transition from a liquid to a gel or solid upon an increase in temperature from 4° C. to 37° C. In some cases, the inverse thermosensitive polymer can be a liquid at 23° C., and undergoes a transition from a liquid to a gel or solid upon an increase in temperature from 23° C. to 37° C.

A number of suitable inverse thermosensitive polymers are known in the art, and suitable for use in conjunction with the methods described herein. In some examples, the inverse thermosensitive polymer can comprise a poly(alkylene oxide), such as a poly(alkylene oxide) block copolymer. In certain examples, the inverse thermosensitive polymer comprises a poloxamer, a poloxamine, or a combination thereof. For example, the inverse thermosensitive polymer can comprise poloxamer 407, poloxamer 188, poloxamer 234, poloxamer 237, poloxamer 338, poloxamine 1107, poloxamine 1307, or a combination thereof. In some embodiments, the inverse thermosensitive polymer can comprise poloxamer 407, poloxamer 188, or a combination thereof.

In certain embodiments, the inverse thermosensitive polymer can comprise (i) from 20% to 40% by weight of a first inverse thermosensitive polymer defined by Formula I below:

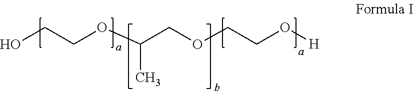

wherein a is an integer from 90 to 110 and b is an integer from 50 to 60, wherein the first inverse thermosensitive polymer has a molecular weight of from 9,500 Da to 15,000 Da, and wherein the first inverse thermosensitive polymer has a polyoxyethylene content of from 70% to 75%; and (ii) from 5% to 50% by weight of a second inverse thermosensitive polymer defined by Formula I below:

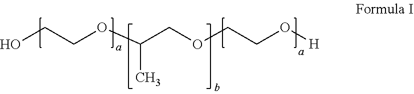

wherein a is an integer from 70 to 90 and b is an integer from 20 to 35, wherein the first inverse thermosensitive polymer has a molecular weight of from 7,500 Da to 10,000 Da, and wherein the first inverse thermosensitive polymer has a polyoxyethylene content of from 78% to 85%.

The system can include a syringe 100 including a body 102 defining a volume containing a biomaterial, e.g., a thermoresponsive polymer. The biomaterial can be introduced into the patient before, during, and/or after the RF ablation procedure. For example, the biomaterial can be introduced at the treatment site before and/or during the ablation procedure to insulate various tissue structures and/or help conduct and direct the electrical signal at select target tissue. In another example, the biomaterial can be delivered to the treatment site before and/or during the ablation procedure to create a physical barrier between the target nerve and the surrounding tissue. In another example, the biomaterial can be introduced at the treatment site during/simultaneous with the RF ablation procedure to facilitate cooling of the treatment site. In another example, the biomaterial can be introduced at the treatment site before, during, and/or after the ablation procedure for drug delivery to the target nervous structure and/or surrounding tissue. Likewise, the biomaterial can be introduced at the treatment site before, during, and/or after the ablation procedure to facilitate additional and/or continued modulation of the target nervous structure.

The biomaterial can be contained in the volume defined by the space between a seal 112 and a delivery tip 130 of the syringe 100. The proximal and distal ends of the syringe 100 are labeled 101A and 101B, respectively, in FIGS. 1A and 1B. As used herein, the terms “distal and “proximal’ are defined with respect to the user and when the device is in use. That is, the term “distal refers to the part or portion further away from the user, while the term “proximal” refers to the part or portion closer to the user, when the device is in use. The syringe 100 can include the delivery tip 130 at the distal end 101B with an opening providing fluid communication from the volume of the body 102. The system can also include a plunger 104 sized and configured to move axially within the volume of the body 102. The axial direction is labeled by dashed line 160 in FIGS. 1A and 1B. When the plunger 104 moves axially within the body 102, the biomaterial liquid can be conveyed through the opening of the delivery tip 130. For example, as the plunger 104 is translated in the axial direction, the biomaterial liquid is pushed by the seal 112 and exits the syringe 100 through the opening of the delivery tip 130. The body 102 and the plunger 104 can include flanges 110 and 108, respectively, which assist a user operating the syringe 100.

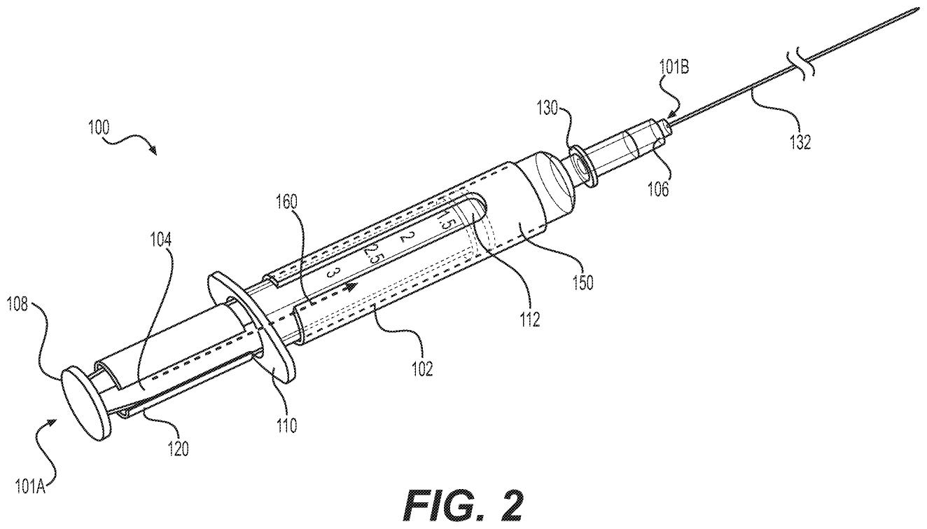

Referring again to FIGS. 1A-2, the syringe 100 can maintain a biomaterial comprising a thermoresponsive polymer liquid, e.g., a poloxamer, at a temperature below its liquid-solid phase transition temperature. As described above, the thermoresponsive polymer can change state in dependence on temperature. Accordingly, in some implementations, the syringe 100 can be configured to keep the thermoresponsive polymer cool such that it is maintained in a liquid state within the syringe 100 prior to delivery to the treatment site. During a medical procedure such as RF ablation, the thermoresponsive polymer such as a poloxamer used for drug delivery can be dispensed from the syringe 100 to the treatment site as a liquid where it then transitions phase as the temperature of the polymer changes. It should be understood that the liquid-solid phase transition temperature is a characteristic of the material, and the syringe 100, needle/cannula 132 and/or introducer 200 can be configured to maintain the thermoresponsive polymer at the desired temperature to prevent solidification of the biomaterial before it reaches the treatment site. The phase transition temperature can range, for example, between about 5° C. and 45° C. for different materials.

The body 102 of the syringe 100 can include a cooling mechanism 150 for maintaining an interior surface of the body 102 at a temperature below the liquid-solid phase transition temperature of the thermoresponsive polymer liquid. As provided in FIGS. 1A and 1B, the cooling mechanism 150 can include a sleeve-like structure that extends circumferentially around the perimeter of the body 102 of the syringe 100 and along a length of the body 102. For example, the sleeve-like structure of the cooling mechanism 150 can be applied over the distal end of the syringe 100 such that the delivery tip 130 extends through an opening at the distal end of the cooling mechanism 150. The body 102 of the syringe 100 may then slide within the elongated annular opening defined by the body of the cooling mechanism 150 to a desired position, where the location of the body 102 with respect to the cooling mechanism 150 is fixed. As illustrated in FIG. 1A, the cooling mechanism 150 defines a sleeve-like structure, wrapping circumferentially around the body 102 of the syringe 100, and covering a portion of the overall length of the body 102, without covering the distal end of the syringe 100. In another example (not shown), the cooling mechanism 150 defines a sleeve-like structure that wrapping circumferentially around the body 102 of the syringe 100 and extends along the entire length of the body 102, without covering the distal end of the syringe 100. In a further example, illustrated in FIG. 1B, the cooling mechanism 150 defines a sleeve-like structure that wraps circumferentially around the body 102 and extends along the entire length of the body 102 of the syringe 100. As provided in FIG. 1B, the distal end of the cooling element 150 extends over at least a portion of the distal end of the body adjacent the delivery tip 130.

The cooling mechanism 150 may include a window 152 to provide visual access by the user to the body 102 and the contents of the plunger 100. For example, the window 152 allows the user to visually inspect the liquid and/or solid state of the polymer contained therein, the location of the plunger 104 within the body 102, and/or any markings on the exterior of the body 102. It is also contemplated that the cooling mechanism 150 will not include a window 152, but rather define an uninterrupted structure extending circumferentially around the entire circumference of the body 102.

The cooling mechanism 150 can be integrally formed with the body 102 of the syringe 100. The cooling mechanism 150 can be removably and/or fixedly coupled to the syringe 100 in a position to cool the thermoresponsive polymer liquid contained therein. The cooling mechanism 150 can be removably and/or fixedly coupled to the body 102 of the syringe 100 by a mechanical and/or chemical fastener (e.g., adhesive). Various mechanical fasteners include, for example, recess or detent and corresponding projection provided between the cooling mechanism 150 and the syringe 100, tapered surface for providing a press fit between the cooling mechanism 150 and the syringe 100, clip, thread, bayonet mount, weld, or any other mechanical fastener. Likewise, the cooling mechanism 150 can be formed around the body 102 such that removal of the cooling mechanism 150 is not possible without damaging the cooling mechanism 150 and/or the syringe 100.

The cooling mechanism 150 can include an insulative material provided around the body 102 of the syringe 100. The insulative material can optionally be attached to one or more portions or areas on the exterior of the body 102 of the syringe 100. The insulative material can optionally be a thin insulating material such as THINSULATE from 3M Company of Maplewood, Minn. Alternatively or additionally, the cooling mechanism 150 and/or body 102 of the syringe 100 can be formed from a double wall vacuum insulated material. Alternatively or additionally, the cooling mechanism 150 can be used to provide active cooling to the syringe 100 and the thermoresponsive polymer. For example, the cooling element 150 can include a cooling pad coupled or attached to the exterior of the body 102 of the syringe 100. For example, the cooling pad component can include, for example, an “instant cold” pad (e.g., water and ammonium nitrate solution the combination of which results in an endothermic reaction causing the cooling pad to rapidly chill) and/or reusable gel cold pad. As described above, the cooling mechanism 150 can be removably coupled to the body 102 of the syringe 100. Similarly, the cooling pad component can be separable from the cooling mechanism 150. For example, the cooling pad component can be replaced in the case of an “instant cold” pad type cooling pad, or the reusable gel cold pad can be chilled for later use.

Alternatively or additionally, the cooling mechanism 150 can include a thermoelectric cooler such as a Peltier cooler. Thermoelectric coolers are known in the art and are therefore not described in further detail herein. Alternatively or additionally, in some implementations, the cooling mechanism 150 can include a chilled water system. The chilled water system can be configured to flow water around the body 102 of the syringe 100. Optionally, the chilled water can be treated with ozone, which can improve heat transfer capability of the cooling mechanism 150.

The cooling mechanism 150 can be used to maintain an interior surface of the body 102 of the syringe 100 at a temperature below about body temperature (e.g., between about 28° C. and about 32° C. external body temperature or between about 35° C. and about 42° C. internal body temperature). In some implementations, an interior surface of the body 102 of the syringe 100 can be maintained at a temperature below about room temperature (e.g., the temperature of a hospital/operating room between about 19° C. and about 25° C.). In some implementations, the syringe 100 and/or the thermoresponsive polymer can be maintained a temperature below about 20° C. In some implementations, the syringe 100 and/or the thermoresponsive polymer can be maintained at a temperature at or below about 4° C. It should be understood that the temperature ranges provided above are provided only as examples. This disclosure contemplates maintaining the syringe 100 and/or thermoresponsive polymer at temperatures other than those provided as examples. As described herein, the syringe 100 can be configured to maintain the thermoresponsive polymer at the material-dependent temperature that prevents solidification.

The cooling mechanism 150 may include one or more temperature sensors along its length for measuring the temperature of the thermoresponsive polymer contained within the syringe 100. The cooling mechanism 150 may also include a temperature indicator for providing information to the user regarding the temperature of the thermoresponsive polymer. The temperature indicator may extend along the length of the cooling mechanism 150 and identify the corresponding temperature of the syringe 100 and/or thermoresponsive polymer at that location. For example, the temperature indicator may identify temperatures along the length of the cooling mechanism 150 so that the user is able to determine if any portion of the thermoresponsive polymer is at a temperature above the phase transition temperature and thereby solidifying within the body 102 of the syringe 100. This is particularly relevant to help the user identify if the thermoresponsive polymer has solidified in the distal portion of the body 102 and is preventing or inhibiting the flow of the polymer through the delivery tip 130.

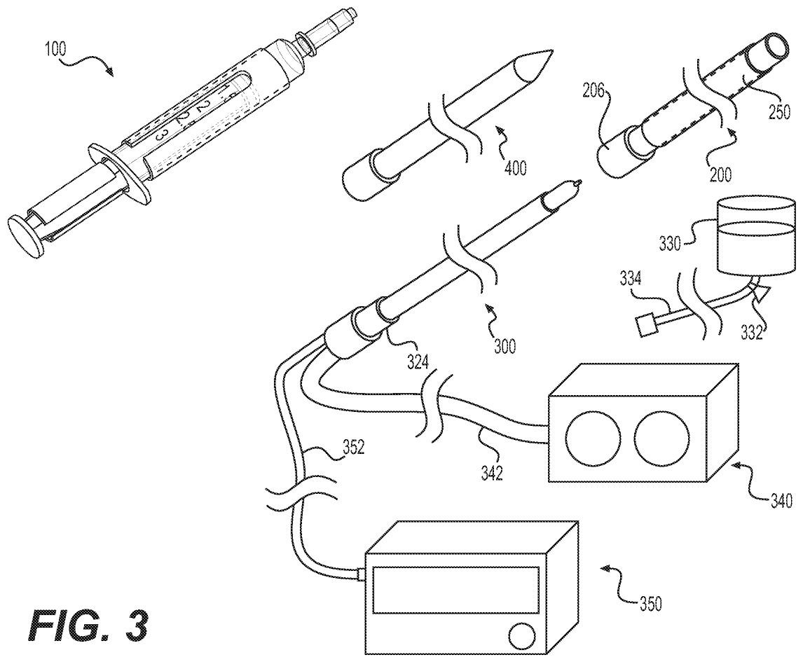

Referring now to FIG. 3, the system can optionally include an elongated introducer cannula 200. In some implementations, RF ablation can be performed on a patient via a catheter, and the introducer cannula 200 can serve as the catheter through which RF ablation is performed, for example, by coupling a cooled RF probe 300 to and/or passing through the introducer cannula 200 to the treatment site. The introducer cannula 200 is used to provide access to the treatment site within the patient's body. The introducer cannula 200 can define proximal and distal ends and a central channel/bore extending therebetween. In use, the proximal end of the introducer cannula 200 is located outside of the patient's body and the distal end of the introducer cannula 200 is located inside the patient's body adjacent the treatment site. The central channel is sized and configured to receive the delivery tip 130 of the syringe 100 therethrough. The introducer cannula 200 can include a single port for accessing the central channel, alternative, the introducer cannula 200 can include separate ports for each of the syringe 100/delivery tip 130/needle 132 and the RF probe 300. As described in more detail below, the elongate shaft 310 of a probe assembly 300 may be introduced to the treatment site through the longitudinal bore of the introducer cannula 200. The introducer cannula 200 may further comprise one or more depth markers in order to enable a user to determine the depth of the distal end of the introducer cannula 200 within a patient's body. Additionally, the introducer cannula 200 may comprise one or more radiopaque markers to ensure the correct placement of the introducer cannula 200 when using fluoroscopic guidance. The introducer cannula 200 may comprise one or more temperature sensors along its length. In such embodiments, the one or more temperature sensors may be placed proximate to the distal end of the introducer cannula 200 so as to enable the one or more temperature sensors to measure the temperature of tissue surrounding the distal end of the introducer cannula 200. The introducer cannula 200 may include multiple temperature sensing elements disposed along the introducer cannula 200 may be used to indicate the size of the lesion as it expands. This may be particularly useful in the treatment of tumor tissue, for example. The introducer cannula 200 may include temperature sensing elements disposed along its central channel for measuring a temperature of the syringe 100, cooling mechanism 150, and/or probe 300. Introducer cannulas 200 may be made of various materials, as is known in the art and, if said material is electrically conductive, the introducer tubes may be electrically insulated along all or part of their length, in order to prevent energy from being conducted to undesirable locations within a patient's body.

Optionally, a second cooling mechanism 250 can be provided to reduce the temperature of the introducer cannula 200. It is contemplated that the second cooling mechanism 250 can be similar to the cooling mechanism 150 as described above. For example, the second cooling mechanism 250 of the introducer cannula 200 can include at least one of an insulative material provided around the introducer cannula 200, a double wall vacuum insulated material, a cooling pad coupled to the introducer cannula 200, a thermoelectric cooler, or a chilled water system for flowing a volume of chilled water around the introducer cannula 200.

As illustrated in FIG. 2, the delivery tip 130 of the syringe 100 can include a needle/cannula 132 for transmitting the biomaterial liquid from the body 102 of the syringe 100, through the introducer cannula 200, and to the treatment site. For example, needle/cannula 132 can include an 18-gauge, 19-gauge, or 20-gauge needle. The introducer cannula 200 into which the delivery tip 130 and needle/cannula 132 is inserted can have a 17-gauge opening. It should be understood that the sizes of the needle and/or introducer cannula described above are provided only as examples. This disclosure contemplates using a needle and/or introducer cannula having sizes other than those provided as examples. The needle/cannula 132 can be flexible or rigid. The needle/cannula 132 can extend at least partially into a central channel of the introducer cannula 200. In one example, the needle/cannula 132 extends only partially into the central channel of the introducer cannula 200 such that the biomaterial liquid passes through the central channel of the introducer cannula 200 to the treatment site. In another example, the needle/cannula 132 extends to the distal end of the introducer 200 and/or beyond the distal end of the introducer 200 such that the liquid biomaterial passes directly from the distal end of the needle/cannula 132 to the treatment site.

The introducer cannula 200 is operable to easily and securely couple with probe assembly 300 and/or syringe 100. For example, the proximal end of the introducer cannula 200 at the introducer hub 206 may be fitted with a connector able to mate reversibly with the hub 324 of probe assembly 300 and/or the delivery tip 130 of the syringe 100. In an example system, the connector between the introducer cannula 200 probe assembly 300 and/or syringe 100 comprises a Luer fitting. Luer fittings are standardized (e.g., International Organization for Standardization (ISO)) fluid connection fittings known in the art and are therefore not described in further detail herein. It is desirable to couple the delivery tip 130 to the proximal end of the introducer cannula 200, for example, when the needle/cannula 132 extends only partially into the introducer cannula 100. A liquid-tight coupling between the delivery tip 130 and the proximal end of the introducer cannula 200 ensures that none of the liquid biomaterial escapes from the proximal end of the introducer cannula 200. Likewise, in an example syringe 100 not including a needle/cannula 132, a liquid-tight coupling between the delivery tip 130 and the introducer cannula 200 allows the introducer cannula 200 to provide fluid communication of the liquid biomaterial between the syringe 100 body 102 and the treatment site while ensuring that none of the liquid biomaterial escapes from the proximal end of the introducer cannula 200.

In an example system, the connector between the introducer cannula 200 and the syringe 100 includes an engagement/locking feature that limits and/or prohibits movement of the plunger 104 and the resulting dispensing of the biomaterial. For example, the connector can include a locking feature is provided at the proximal end of the introducer cannula 200 and couples with a corresponding engagement feature 106 provided at the distal end of the syringe 100. Engagement between the engagement/locking feature in the hub 206 of the introducer cannula 200 and the engagement features 106 of the syringe 100 can be used to limit and/or prohibit movement of the plunger 104 and the resulting dispensing of the biomaterial. The engagement/locking feature is movable between unlocked and locked configurations. As used herein, when in the unlocked configuration, engagement feature 106 and the corresponding engagement/locking feature in the hub 206 are not coupled. In the unlocked configuration, axial movement of the plunger 104 within the volume of the body 102 is prohibited. On the other hand, when in the locked configuration, the engagement feature 106 and the corresponding engagement/locking feature in the hub 206 are coupled, and axial movement of the plunger 104 within the volume is permitted. Optionally, the engagement feature 106 and corresponding engagement feature in the hub 206 can be male and female Luer fittings provided at the distal end of the syringe 100 and the proximal end of the introducer cannula 200, respectively. Rotational movement between the male and female Luer fittings results in secured coupling between the introducer cannula 200 and the syringe 100. It should be understood that Luer fittings are provided only as examples of engagement features. This disclosure contemplates that the engagement feature 106 and corresponding engagement feature in the hub 206 can be fittings other than Luer fittings. For example, the engagement feature 106 and corresponding engagement feature in the hub 206 can optionally include at least one of a recess or detent provided in one of the delivery tip 130 or hub 206 and a corresponding a projection provided in the opposing structure, clip, thread, bayonet mount, or any other mechanical fastener/coupling capable for providing a secure and generally leak-proof coupling and locking feature between the syringe 100 and the introducer cannula 200. This disclosure contemplates that feature 106 and corresponding engagement feature in the hub 206 can be configured to couple and allow for relative movement between the locked and unlocked configurations. It should be understood that the design of the engagement feature 106 and corresponding engagement feature in the hub 206 should not be limited by the examples provided above.

Optionally, and as shown in FIGS. 1A and 1B, the system can include a plunger restraint mechanism 120 coupled to the plunger 104. The restraint mechanism 120 can be configured to restrict (e.g., physically restrict) axial movement of the plunger 104 and delivery of the thermoresponsive polymer. The restraint mechanism 120 can be removed by a user to permit axial movement of the plunger 104 within the body 102 of the syringe 100. As illustrated in FIGS. 1A and 1B, the restraint mechanism 120 is in the form of an elongated c-shaped clip that is received over the stem portion of the plunger 104 between flange 108 of the plunger 104 and flange 110 of the body 102. Opposing sides of the restraint mechanism 120 can be flexed to increase the gap therebetween and remove the restraint mechanism 120 from the plunger 104 stem. As illustrated in FIGS. 1A and 1B, the length of the plunger restraint mechanism 120 corresponds to the location of the seal 112 within the body 102, where the location of the seal 112 is determinative of the volume of the biomaterial included in the body 102. It is contemplated that different lengths of restraint mechanisms 120 can be provided to accommodate different seal 112 locations, i.e., different volumes of biomaterial. In another example (not shown), the plunger restraint mechanism 120 is in the form of a break-away/tear-away tab or barrier that restricts axial movement of the plunger 104.

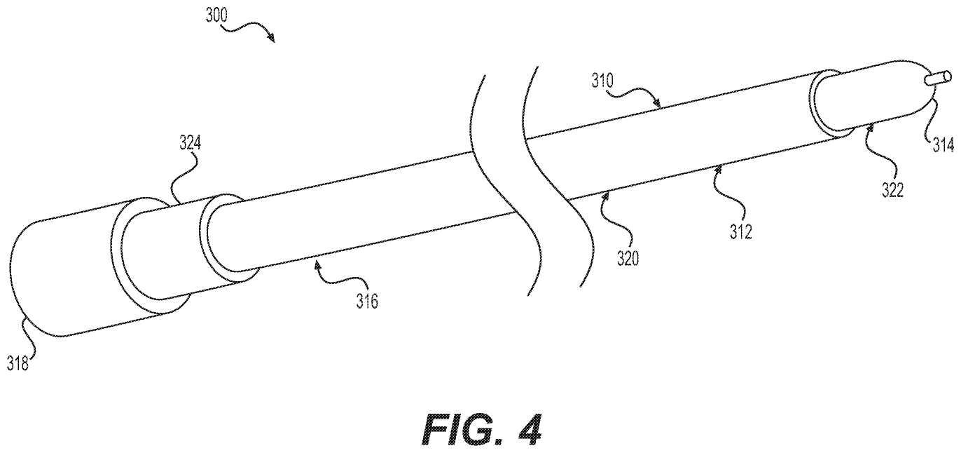

As provided above, the temperature-controlled syringe 100 described herein may be used with systems providing RF treatment of bodily tissue, in particular, cooled probes including some form of temperature and/or impedance monitoring. An example probe 300 is provided in FIGS. 3 and 4. The probe 300 includes an elongate member, comprising an elongated shaft 310, a distal tip region 312 comprising one or more energy delivery devices, a distal end 314, a proximal region 316, and a proximal end 318. With reference to FIG. 3, an embodiment of a system suitable for use with probe 300 may comprise one or more of: one or more introducer cannula 200; one or more dispersive return electrodes (not shown); one or more sources of cooling, for example pump 340; one or more energy sources, for example generator 350 and one or more connecting means, for example tube 342 and/or cable 352

Probe 300 comprises an electrically insulated portion 320 and an electrically exposed conductive portion 322. Electrically exposed conductive portion 322 may also be referred to as an active electrode and is an example of an “energy delivery portion” of the probe 300. The proximal region 316 of probe 300 comprises a hub 324. Hub 324 is structured to operatively connect other devices, such as connector cables, cannulae, tubes, or other hubs, for example, to probe 300. For example, probe 300 may be coupled to an energy source and/or to a source of cooling via respective connecting means (for example, an electrical cable and/or flexible tubing) which may be associated with hub 324. Hub 324 may also serve as a handle or grip for probe 300. Hub 324 may be manufactured from a number of different materials, including, but not limited to, plastics, polymers, metals, or combinations thereof. Furthermore, hub 324 may be attached to probe 300 by a number of different means. For example, in one embodiment, hub 324 may be made from polypropylene, and may be attached to probe 300 by insert molding.

The size of probe 300 may vary, depending upon which of the method embodiments, described herein below, are used. In some examples, the length from distal end 314 to proximal end 318 of probe 300 may be between about 5 cm and about 40 cm and the outer diameter of shaft 310 may be between about 0.65 mm and about 2.00 mm (between about 20 G and about 12 G). In one specific example, the length of the probe may be about 7.5 cm and the outer diameter may be about 1.5 mm. Furthermore, the size and shape of active electrode 322 may vary, as is further described in U.S. patent application Ser. No. 11/457,697 (issued as U.S. patent Ser. No. 10/206,739), previously incorporated herein by reference. For example, in some embodiments, active electrode 322 may be between about 2 mm and about 8 mm in length. In other embodiments, active electrode 322 may comprise substantially only the distal face of probe 300.

In some embodiments, electrically insulated portion 320 may be formed by coating a portion of shaft 310 with an electrically insulative coating, covering, or sheathing. For example, in one particular embodiment, shaft 310 of probe 300 may be fabricated from a biocompatible metal or alloy, for example stainless steel, which may be overlaid in part by an insulating coating, for example polytetrafluoroethylene (PTFE). In other embodiments, shaft 310 may be fabricated from another metal, such as nitinol or titanium, and/or the insulating coating may comprise a different electrically insulating material, including but not limited to polyethylene terephthalate (PET). In other embodiments, other metals or electrically insulating materials may be used.

The probe 300 is structured such that it may be cooled by the internal circulation of a cooling fluid. Such a configuration, whereby a cooling medium does not exit from a distal region 312 of probe 300, may be referred to as an internally-cooled probe. The cooling fluid may be any fluid suitable for removing heat from probe 300 during surgery, for example water. Other examples of cooling fluid include, but are not limited to, liquid nitrogen and saline. Furthermore, the fluid may be at any temperature suitable for removing heat from the probe during surgery, for example between about 0° C. and about 25° C. More specifically, the temperature of the fluid may be at about room temperature (21° C.), about 4° C., or about 0° C., depending on the application.

The fluid may be delivered or circulated at a wide range of flow-rates. An appropriate flow-rate may be determined or calculated based on a number of factors, including the conductivity and heat capacity of probe 300, the cooling fluid and/or the tissue, and the desired temperature of distal end 314 of probe 300, among other factors. In some embodiments, the fluid may be delivered at between about 10 ml/min and about 30 ml/min.

As mentioned hereinabove, one or more fluids may be delivered from a reservoir to the probe 300 for the purposes of cooling the probe 300. The fluid(s) may be delivered to the probe 300 via a number of means, and the invention is not limited in this regard. For example, in one embodiment and with reference to FIG. 3, the reservoir of fluid may comprise a container, for example an intravenous (IV) bag 330, which is elevated above the patient. Tubing 332, for example clear plastic flexible tubing, may be used to connect the reservoir to an inlet in probe 300. A valve 334 may be placed at the junction of the container and the tubing (or at some other location between the container and the probe), such that when the valve 334 is opened, gravity may cause fluid to flow towards probe 300. After circulation within probe 300, fluid may exit probe 300 via tubing, which may drain into another reservoir, for example a second IV bag, or into a sink or other drain. In another embodiment, at least one pump may be used to deliver fluid to the probe 300.

The probe 300 can also include a peristaltic pump 340 operatively connected to a reservoir of fluid and configured to circulate the chilled fluid (e.g., deionized (DI) water). The reservoir of fluid may be an IV bag, a polypropylene vial or burette, or another container, for example. The pump 340 may pump the fluid from the reservoir to an inlet in probe 300. After circulating in probe 300, the fluid may exit the probe through an outlet in probe 300 and may flow through a tube to either the same or a different reservoir or, alternatively, to an alternate location as described above. A heat sink, heat exchanger, or other cooling source such as a refrigerant chiller may be used to cool the fluid after exiting the probe 300. A second pump, gravity, or a source of suction, for example, may assist in drawing the fluid out of the probe 300. The use of other types of pumps to supply a cooling fluid to and return a cooling fluid from the probe 300 is contemplated including, for example, a centrifugal pump or a piston pump. Further details regarding the cooling source are provided in U.S. patent application Ser. No. 11/105,527 (filed on Apr. 14, 2005) and Ser. No. 10/864,410 (filed on Dec. 10, 2005).

By leaving the probe 300 coupled to the introducer cannula 200 and running the pump 340 before delivering the thermoresponsive polymer within the syringe 100 via the cannula 200, the probe 300 can be used to pre-cool the introducer cannula 200 and/or treatment site by circulating chilled liquid through the probe 300. In some implementations, the probe 300 can be used to maintain the introducer cannula 200 at a temperature below the liquid-solid phase transition temperature of the thermoresponsive polymer.

Probe 300 is structured to be operatively connected to an energy source, for example a generator 350. The connecting means for connecting probe 100 to generator 350 may comprise any component, device, or apparatus operable to make one or more electrical connections, for example an insulated wire or cable. In one embodiment, the connecting means comprises an electrical cable 352 terminating at hub 324 as well as a connector at a proximal end thereof. Cable 352 may be operable to couple to energy source 350 directly or indirectly, for example via an intermediate cable. At least one wire or other electrical conductor associated with cable 352 may be coupled to a conductive portion of shaft 310, for example by a crimp or solder connection, in order to supply energy from energy source 350 to shaft 310. In one specific embodiment, a 4-pin medical connector is used to connect cable 352 to an intermediate cable (not shown), which may be further attached to a 14-pin connector capable of being automatically identified when connected to generator 350. Further details regarding such an embodiment are disclosed in U.S. patent application Ser. No. 10/122,413 (filed on Apr. 16, 2002), incorporated herein by reference.

Generator 350 may produce various types of energy, for example microwave or radio-frequency electrical energy. In some embodiments, generator 350 produces radiofrequency electrical current, having a frequency of between about 10 kHz and about 1000 kHz, at a power of between about 1 W and about 50 W. An example of an RF generator that may be used as part of a system of the present invention is the Pain Management Generator (PMG) of Baylis Medical Company Inc. (Montreal, QC, Canada). Further details regarding embodiments of energy sources are disclosed in U.S. patent application Ser. No. 11/457,697 (issued as U.S. patent Ser. No. 10/206,739), previously incorporated herein by reference.

As illustrated in FIGS. 3 and 4, the temperature-controlled syringe 100 and probe 300 described herein when used in conjunction with one or more introducer cannulas 200, may further comprise one or more stylets 400. The stylet 400 may have a beveled tip to facilitate insertion of the introducer cannula 200 into a patient's body. Various forms of stylets 400 are well known in the art and the present invention is not limited to include only one specific form.

Optionally, the system can include a motor. The motor can be operably coupled to the syringe 100 and/or the cooling unit 150/250. The motor can be configured to control the flow of the biomaterial from the syringe 100 and/or the flow of cooling fluid in the cooling unit 150/250.

The system can optionally include a tagging system. In some implementations, the tagging system includes an electronically scannable code including, but not limited to, a bar code or QR code. For example, the electronically scannable code can be provided on the body 102 of the syringe 100 such that the electronically scannable code can be read by the system prior to delivering the biomaterial. The electronically scannable code can encode information including, but not limited to, the temperature at which to maintain the biomaterial and/or the flow rate. Such information can be used by the system for controlling the motor, cooling unit 150/250, and/or cooled RF probe 300. Alternatively or additionally, in some implementations, the system can optionally include a tagging system configured to determine the unlocked and locked configurations of the syringe 100 and introducer cannula 200. For example, the tagging system can include a radiofrequency identification (RFID) integrated circuit. The RFID integrated circuit can be provided on portions of the engagement features 106, 206 described above. For example, each of the introducer cannula 200 and the syringe 100 can include a respective locking portion. Optionally, the corresponding locking portions can be provided on the corresponding engagement features 106, 206 described above. The RFID integrated circuit can confirm alignment of a locking portion of the introducer cannula 200 with a corresponding locking portion of the syringe 100. When the RFID integrated circuit determines that the locking portions of the introducer cannula and syringe are not in alignment, the introducer cannula 200 and syringe 100 are in the unlocked configuration. As described above, the syringe and introducer cannula engagement features 106, 206 are not coupled in the unlocked configuration and axial movement of the plunger 104 is prohibited. When the RFID integrated circuit determines that the locking portions of the introducer cannula and syringe are in alignment, the introducer cannula 200 and syringe 100 are in the locked configuration. As described above, the syringe and introducer cannula engagement features 106, 206 are coupled in the locked configuration and axial movement of the plunger 104 is permitted. Optionally, the RFID integrated circuit can provide information to the system, and such information can be used for controlling the motor and/or cooling unit 150/250. In some implementations, such information can be used to control lights or other indicators provided by the system to facilitate manual or automatic control. It should be understood that the tagging systems described above are provided only as examples. This disclosure contemplates using tagging systems other than electronically scannable codes and/or RFID integrated circuits.

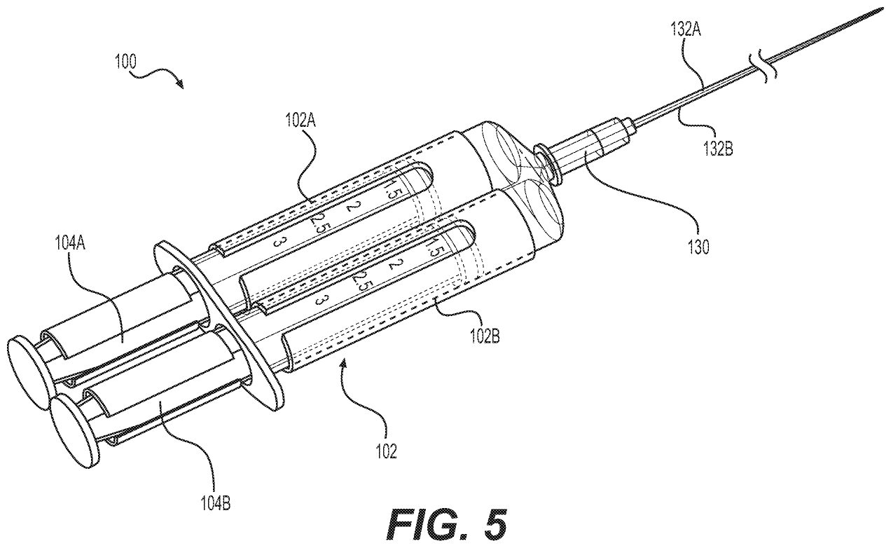

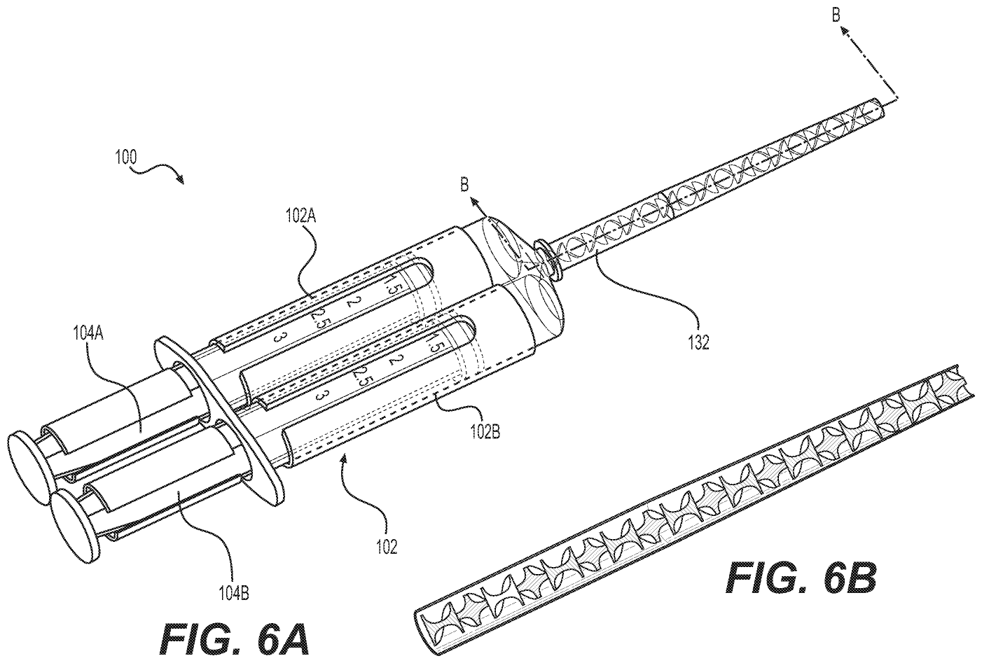

As illustrated in FIGS. 5-6B, in some implementations, the syringe 100 can have a double barrel configuration. A double barrel configuration can be used to combine or mix a crosslinkable biomaterial at the treatment site. As described in more detail below, the crosslinkable biomaterial can be formed from a first precursor and a second precursor which react in situ to form a polymeric matrix. Accordingly, the first precursor and the second precursor must be separately stored prior to being combined at the treatment site. As provided in FIGS. 5-6B, the body 102 of the double barrel syringe 100 can define a second volume 102a separate from the main volume 102b and corresponding plungers first plunger 104a and a second plunger 104b. The main volume 102a can be configured to contain a first precursor and the second volume 102b can be configured to contain a second precursor. Optionally, the syringe 100 may include a cooling mechanism 150 for maintaining the first and second precursors at a desired temperature. Illustrated in FIG. 5, the delivery tip 130 can include an elongated needle 132 defining a first bore 132a and a separate second bore 132b, where the first bore 132a is in fluid communication with the main volume 102a of the syringe 100 and the second bore 132b is in fluid communication with the second volume 102b of the syringe 100. Accordingly, a first precursor and a second precursor transmitted through the needle 132 first contact/combine as they pass through a distal end of the needle 132. Alternatively, as illustrated in FIG. 6A, the delivery tip 130 and/or needle/cannula 132 can include a mixing chamber wherein the first precursor and the second precursor combine before exiting the needle/cannula 132 and/or introducer cannula 200. The mixing chamber can be positioned at any suitable point along the length of the delivery tip 130 and/or needle/cannula 132. In some examples, the mixing chamber is positioned entirely within the delivery tip 130, such that the first and second precursors are combined before entering the needle/cannula 132. In another example, the mixing chamber is positioned within the distal region of the needle/cannula 132. In a further example, as illustrated in FIG. 6A, the mixing chamber is positioned along a majority of the length of the needle/cannula 132.

The mixing chamber can include one or more fluid channels configured to mix two solutions flowing into the mixing channel from the first body 102a and the second body 102b. The mixing chamber can adopt a variety of geometries, based on for example the solutions to be mixed and the length of the mixing chamber. For example, the mixing chamber can be configured to mix (e.g., to render homogeneous) two solutions which flow through the mixer. The mixing chamber can be, for example, a channel (e.g., a serpentine or tortuous channel, or a channel containing one or more protrusions) which induces turbulent flow so as to mix the fluids. As illustrated in FIGS. 6A and 6B, the mixing chamber can include a helical mixing path, the mixing path is defined by helixes that form 180° turns within the central lumen of the needle/cannula 132. Each turn mixes the two precures about 50%, such that a homogenous mixture exits the needle/cannula 132 and is introduced at the treatment site. In another example (not shown), a barrier separating the main volume from the second volume can be opened such that contents of the main volume and the second volume combine within the syringe 100 before passing through the delivery tip 130.

Suitable precursor molecules can be selected in view of the desired properties of the crosslinkable biomaterial and resultant polymeric matrix. In some cases, the crosslinkable biomaterial comprises one or more oligomeric or polymeric precursor molecules. For example, precursor molecules can include, but are not limited to, polyether derivatives, such as poly(alkylene oxide)s or derivatives thereof, polysaccharides, peptides, and polypeptides, poly(vinyl pyrrolidinone) (“PVP”), poly(amino acids), and copolymers thereof.

The precursor molecules can further comprise one or more reactive groups. Reactive groups are chemical moieties in a precursor molecule which are reactive with a moiety (such as a reactive group) present in another precursor molecule to form one or more covalent and/or non-covalent bonds. Examples of suitable reactive groups include, but are not limited to, active esters, active carbonates, aldehydes, isocyanates, isothiocyanates, epoxides, alcohols, amines, thiols, maleimides, groups containing one or more unsaturaturated C—C bonds (e.g., alkynes, vinyl groups, vinylsulfones, acryl groups, methacryl groups, etc.), azides, hydrazides, dithiopyridines, N-succinimidyl, and iodoacetamides. Suitable reactive groups can be incorporated in precursor molecules to provide for crosslinking of the precursor molecules.

In some embodiments, one or more of the precursor molecules comprises a poly(alkylene oxide)-based oligomer or polymer. Poly(alkylene oxide)-based oligomer and polymers are known in the art, and include polyethylene glycol (“PEG”), polypropylene oxide (“PPO”), polyethylene oxide-co-polypropylene oxide (“PEO-PPO”), co-polyethylene oxide block or random copolymers, poloxamers, meroxapols, poloxamines, and polyvinyl alcohol (“PVA”). Block copolymers or homopolymers (when A=B) may be linear (AB, ABA, ABABA or ABCBA type), star (AₙB or BAₙC, where B is at least n-valent, and n is an integer of from 3 to 6) or branched (multiple A's depending from one B). In certain embodiments, the poly(alkylene oxide)-based oligomer or polymer comprises PEG, a PEO-PPO block copolymer, or combinations thereof.

In some embodiments, one or more of the precursor molecules is defined by Formula I or Formula II:

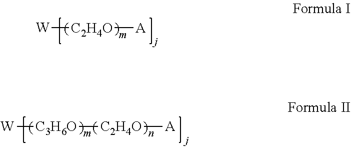

wherein:

W is a branch point;

A is a reactive group (e.g., a nucleophilic group or a conjugated unsaturated group);

m and n are integers of from 1 to 500 (e.g., an integers of from 1 to 200); and

j is an integer greater than 2 (e.g., an integer of from 2 to 8).

In some embodiments, one or more of the precursor molecules comprises a biomacromolecule. The biomacromolecule can be, for example, a protein (e.g., collagen) or a polysaccharide. Examples of suitable polysaccharides include cellulose and derivatives thereof, dextran and derivatives thereof, hyaluronic acid and derivatives thereof, chitosan and derivatives thereof, alginates and derivatives thereof, and starch or derivatives thereof. Polysaccharides can derivatized by methods known in art. For example, the polysaccharide backbone can be modified to influence polysaccharide solubility, hydrophobicity/hydrophilicity, and the properties of the resultant polymeric matrix formed from the polysaccharide (e.g., matrix degradation time). In certain embodiments, one or more of the precursor molecules comprises a biomacromolecule (e.g., a polysaccharide) which is substituted by two or more (e.g., from about 2 to about 100, from about 2 to about 25, or from about 2 to about 15) reactive groups (e.g., a nucleophilic group or a conjugated unsaturated group).

In some cases, the crosslinkable biomaterial can comprise a first precursor molecule which comprises an oligomer or polymer having one or more first reactive groups, each first reactive group comprising one or more pi bonds, and a second precursor molecule comprises an oligomer or polymer having one or more second reactive groups, each second reactive group comprising one or more pi bonds. The first reactive group can be reactive (e.g., via a Click chemistry reaction) with the second reactive group, so as to form a covalent bond between the first precursor molecule and the second precursor molecule. For example, the first reactive group and the second reactive group undergo a cycloaddition reaction, such as a [3+2] cycloaddition (e.g., a Huisgen-type 1,3-dipolar cycloaddition between an alkyne and an azide) or a Diels-Alder reaction.

In some cases, the crosslinkable biomaterial can comprise a first precursor molecule which comprises an oligomer or polymer having one or more nucleophilic groups (e.g. amino groups, thiol groups hydroxy groups, or combinations thereof), and a second precursor molecule which comprises an oligomer or polymer having one or more conjugated unsaturated groups (e.g., vinyl sulfone groups, acryl groups, or combinations thereof). In such cases, the first precursor molecule and the second precursor molecule can react via a Michael-type addition reaction. Suitable conjugated unsaturated groups are known in the art, and include those moieties described in, for example, U.S. Patent Application Publication No. US 2008/0253987 to Rehor, et al., which is incorporated herein by reference in its entirety.

In certain embodiments, the crosslinkable biomaterial can comprise a first precursor molecule and a second precursor molecule. The first precursor molecule comprises a poly(alkylene oxide)-based oligomer or polymer having x nucleophilic groups, wherein x is an integer greater than or equal to 2 (e.g., an integer of from 2 to 8, or an integer of from 2 to 6). The poly(alkylene oxide)-based polymer can comprise, for example, poly(ethylene glycol). The nucleophilic groups can be selected from the group consisting of sulfhydryl groups and amino groups. The first precursor molecule can have a molecular weight of from about 1 kDa to about 10 kDa (e.g., from about 1 kDa to about 5 kDa). In some embodiments, the first precursor molecule comprises pentaerythritol poly(ethylene glycol)ether tetrasulfhydryl.

The second precursor molecule can comprise a biomacromolecule having y conjugated unsaturated groups, wherein y is an integer greater than or equal to 2 (e.g., an integer of from 2 to 100, or an integer of from 2 to 25). The biomacromolecule can comprise a polysaccharide, such as dextran, hyaluronic acid, chitosan, alginate, or derivatives thereof. The conjugated unsaturated groups can be selected from the group consisting of vinyl sulfone groups and acryl groups. The second precursor molecule can have a molecular weight of from about 2 kDa to about 250 kDa (e.g., from about 5 kDa to about 50 kDa). In some embodiments, the second precursor molecule comprises dextran vinyl sulfone.

In some embodiments, the in situ crosslinking of the precursor molecules takes place under basic conditions. In these embodiments, the crosslinkable biomaterial can further include a base to activate the crosslinking of the precursor molecules. A variety of bases comply with the requirements of catalyzing, for example, Michael addition reactions under physiological conditions without being detrimental to the patient's body. Suitable bases include, but are not limited to, tertiary alkyl-amines, such as tributylamine, triethylamine, ethyldiisopropylamine, or N,N-dimethylbutylamine. For a given composition (and mainly dependent on the type of precursor molecules), the gelation time can be dependant on the type of base and of the pH of the solution. Thus, the gelation time of the composition can be controlled and adjusted to the desired application by varying the pH of the basic solution.

In some embodiments, the base, as the activator of the covalent crosslinking reaction, is selected from aqueous buffer solutions which have their pH and pK value in the same range. The pK range can be between 9 and 13. Suitable buffers include, but are not limited to, sodium carbonate, sodium borate and glycine. In one embodiment, the base is sodium carbonate.

An example method of delivering a biomaterial to a treatment site is also described generally as follows. This disclosure contemplates using the temperature-controlled systems described above with respect to FIGS. 1-4 to deliver the biomaterial liquid to the treatment site. However, it is contemplated that the double barrel syringe 100 depicted in FIGS. 5-6B for use with a crosslinkable biomaterial may also be used.

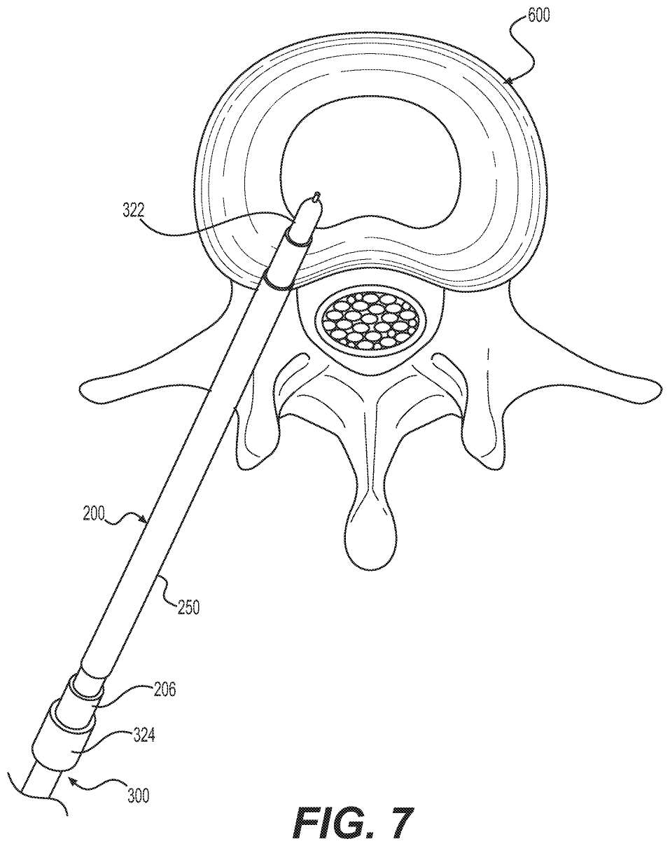

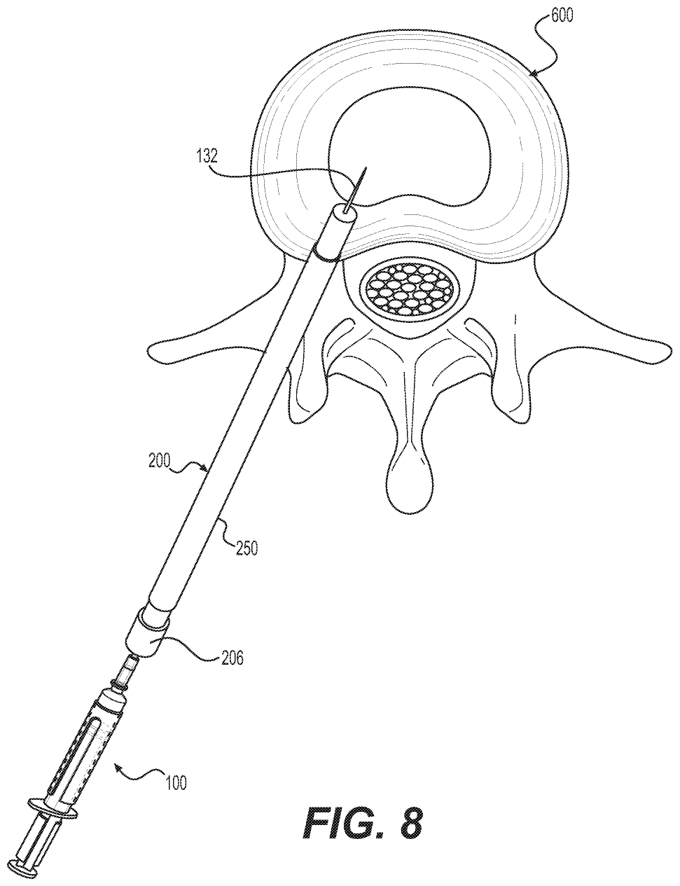

With a patient lying on a radiolucent table, fluoroscopic guidance can optionally be used to percutaneously insert an introducer canula (e.g., the introducer cannula 200 shown in FIG. 3) into a body of a patient, where a distal end of the introducer cannula 200 is located inside the body of the patient and proximate a treatment site and a proximal end of the introducer cannula 200 is located outside the body of the patient (e.g., as shown in FIGS. 7 and 8). For example, as illustrated in FIG. 7, the introducer cannula 200 is inserted into the posterior of an intervertebral disc 600. In addition to fluoroscopy, other aids, including but not limited to impedance monitoring and tactile feedback, may be used to assist a user to position the introducer or probe assemblies within the patient's body. Impedance monitoring allows the user to distinguish between tissues by monitoring impedance as a device is inserted into the patient's body. With respect to tactile feedback, different tissues may offer different amounts of physical resistance to an insertional force. This allows a user to distinguish between different tissues by feeling the force required to insert a device through a given tissue. If more than one probe 300 assemblies are needed to facilitate the treatment, additional introducer cannulas 200 can be inserted into the patient in the same manner, and the stylets 400 removed.

As described herein, the introducer cannula can be used as the catheter through with RF ablation is performed. Probe assemblies 300 are inserted into each of introducers placing electrodes 322 proximate the tissue and/or nervous structure being treated. Once in place, a stimulating electrical signal may be emitted from either of electrodes 322 to a dispersive electrode or to the other electrode 322 (if two are used). This signal may be used to stimulate sensory nerves where replication of symptomatic pain would verify proximity/placement at the target nervous structure that the disc is pain-causing. A different signal may be used to stimulate motor nerves where a motor reaction indicates unsafe proximity to motor nerves that should not be heated. As is well known in the art, various frequencies and voltages can be used to stimulate both sensory and motor nerves. Observation of said stimulation can take the form of visual, sensory, mechanical, or electrical detection of muscle activity, or the form of sensory or electrical detection of nociceptive or other sensory neural activity (e.g. temperature sensation). The electrical energy (“stimulation energy”) applied during this step is beneficially capable of eliciting a response from a neural structure without damaging the neural structure. Using this step, it can be determined whether a target nerve or nerves has a function that would contraindicate its ablation or functional alteration. In one embodiment, the lack of a contraindication would lead to the step of delivering energy, whereas the presence of a contraindication would lead back to the step of inserting one or more probe assemblies, whereby the step of inserting a probe assembly includes modifying the position of a probe assembly within the body. Furthermore, in some embodiments, a method of this aspect of the present invention may comprise a step of stimulating neural tissue after a treatment procedure in order to determine the effectiveness of the treatment procedure.

Probe assemblies 300 are connected to an RF generator 350 as well as to peristaltic pumps 340 to cool distal tip regions 312 of the probe 300. Radio frequency energy is delivered to electrodes 322 and the power is altered according to the temperature measured by a temperature sensing element provided on the tip of electrode 322 such that a desired temperature is reached between at the distal tip region 312. During the procedure, a treatment protocol such as the cooling supplied to the probe assemblies 300 and/or the power transmitted to the probe assemblies 300 may be adjusted in order to maintain a desirable treatment area shape, size and uniformity. The cooling devices may be independently controlled to alter the rate of cooling to the electrode 322. Following treatment, energy delivery and cooling are stopped and probe assemblies 106 are removed from introducers.

The method can also include inserting a delivery tip of a syringe (e.g., the syringe 100 shown in FIGS. 1A-2 and 5-6B) into the proximal end of the introducer cannula 200. As described herein with respect to FIGS. 1A-2, a biomaterial such as a stimuli-responsive biomaterial, e.g., a thermoresponsive polymer comprising a poloxamer, can be contained within a body of the syringe. Likewise, as described with respect to FIGS. 5-6B, a crosslinkable biomaterial can be contained with the body of the syringe. As described above, the biomaterial can be used before, during and after an RF ablation procedure to insulate various tissue structures, conduct and direct the electrical signal to target tissue, to facilitate cooling of the treatment site, for drug delivery to the target nervous structure and/or surrounding tissue, to create a physical barrier between the target nerve and the surrounding tissue, and/or to facilitate additional and/or continued modulation of the target nervous structure. Accordingly, in an example treatment method, with the probe 300 removed from the introducer cannula 200, the needle/cannula 132 is inserted into the introducer cannula 200 (FIG. 8), the plunger is depressed, and the biomaterial is introduced to the treatment site. In another example, with the probe 300 maintained within the introducer cannula 200, the needle/cannula 132 is inserted into the introducer cannula 200, the plunger depressed, and the biomaterial is introduced into the treatment site.

Optionally, the delivery tip 130 can be coupled to the proximal end of the introducer cannula 200 to provide a liquid-tight fit. However, where the syringe 100 does not include a needle/cannula 132 the delivery tip 130 is coupled to the proximal end of the introducer cannula 200 to provide a liquid-tight fit. Optionally, the method can include moving the introducer cannula 200 and syringe 100 from a locked to an unlocked configuration, for example, using engagement features (e.g., the engagement features 106, 206 shown in FIGS. 1A-2) to allow depression of the plunger 104.

Where the biomaterial comprises a thermoresponsive polymer, the method can further include maintaining the thermoresponsive polymer at a temperature below its liquid-solid phase transition temperature. The plunger 104 of the syringe 100 is depressed to advance the thermoresponsive polymer in its liquid state through a delivery tip 130 of the syringe 100 and to the treatment site. Following injection, the liquid composition can form a polymeric matrix at (e.g., a solid or gel) at the treatment site upon injection into a physiological environment. The polymeric matrix can substantially surround the lesion, thereby modulating neural regeneration at the lesion site. For example, the polymeric matrix can limit or prevent macrophages from accessing the lesion site. Macrophages secrete growth factors that drive neurogenesis at the proximal end of the nerve. By limiting and/or preventing the ability of macrophages to access the lesion site, the quantity of neural growth factors reaching the lesion site (and by extension nerve growth at the proximal end of the nerve) can be controlled. If desired, active agents to control nerve growth can also be incorporated in the polymeric matrix. In these embodiments, the polymeric matrix can function as a depot, providing for controlled, local delivering these active agents at the lesion site. Thus, neural regeneration at the lesion site can be guided and controlled using a polymeric matrix, thereby prolonging the pain-relieving effects of the neural ablation procedures.

Where the biomaterial comprises a thermoresponsive polymer, the method can include cooling the syringe 100, the introducer cannula 200, and/or thermoresponsive polymer to a temperature below the liquid-solid phase transition temperature of the thermoresponsive polymer. This can be accomplished, for example, using a cooling mechanism, e.g., the cooling mechanism 150 shown in FIGS. 1A-2). The method can be performed during an RF ablation procedure. In this implementation, the method can optionally include cooling the introducer cannula using a cooled RF probe (e.g., the cooled RF probe 300 shown in FIG. 2). Alternatively or additionally, this can be accomplished, for example, using a cooling mechanism (e.g., the second cooling mechanism 250 shown in FIG. 4). Optionally, the introducer cannula can be cooled to a temperature below the liquid-solid phase transition temperature of the thermoresponsive polymer.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A temperature-controlled system for delivering a stimuli-responsive biomaterial to a treatment site, the system comprising:
a syringe including:
a body defining a volume for containing a thermoresponsive polymer liquid, a distal end of the body includes a delivery tip, and
a plunger sized and configured to move within the volume of the body to convey the thermoresponsive polymer liquid through the delivery tip,
wherein the syringe maintains the thermoresponsive polymer liquid at a temperature below its liquid-solid phase transition temperature.

2. The system of claim 1, wherein the body of the syringe includes a cooling mechanism for maintaining an interior surface of the body at a temperature below the liquid-solid phase transition temperature of the thermoresponsive polymer liquid.

3. The system of claim 2, wherein the cooling mechanism includes at least one of an insulative material provided around the body of the syringe, a cooling pad coupled to the exterior of the body of the syringe, a chilled water system where a volume of chilled water is flowed around the body of the syringe, and a thermoelectric cooler.

4. The system of claim 2, wherein the body of the syringe is formed from a double wall vacuum insulated material.

5. The system of claim 1, further comprising:
an elongated introducer cannula including a central channel sized and configured to receive the delivery tip of the syringe, and
a cooled radiofrequency (RF) probe sized for insertion into the central channel of the introducer cannula, the cooled RF probe being configured to circulate water at a temperature that maintains the introducer cannula at a temperature below the liquid-solid phase transition temperature of the thermoresponsive polymer liquid.

6. The system of claim 5, wherein the introducer cannula includes at least one of an insulative material provided around the introducer cannula, a double wall vacuum insulated material, a cooling pad coupled to the introducer cannula, a thermoelectric cooler, or a chilled water system for flowing a volume of chilled water around the introducer cannula.

7. The system of claim 1, wherein an interior surface of the body of the syringe is maintained at a temperature below about body temperature, wherein the interior surface of the body of the syringe is maintained at a temperature between about 28° C. and about 42° C.

8. The system of claim 1, wherein an interior surface of the body of the syringe is maintained at a temperature below about room temperature, wherein the interior surface of the body of the syringe is maintained at a temperature between about 19° C. and about 25° C.

9. The system of claim 1, wherein the syringe or the thermoresponsive polymer is maintained at a temperature at or below about 4° C.

10. The system for delivering a biomaterial to a radio frequency ablation treatment site, the system comprising:
a syringe including:
a body defining a volume for containing a biomaterial, a distal end of the body includes a delivery tip, and
a plunger sized and configured to move within the volume of the body to convey the biomaterial through the delivery tip, and
an elongated introducer cannula including:
a central channel sized and configured to receive the delivery tip of the syringe,
an engagement feature provided at a proximal end of the introducer cannula for coupling with a corresponding engagement feature provided at a distal end of the syringe,
wherein the syringe and introducer cannula are movable between an unlocked and locked configuration, where in the unlocked configuration the syringe and introducer cannula engagement features are not coupled, and where in the locked configuration the syringe and introducer engagement features are coupled.

11. The system of claim 10, wherein in the unlocked configuration axial movement of the plunger within the volume of the body is prohibited, preventing flow of the biomaterial through the delivery tip,
wherein in the locked configuration axial movement of the plunger within the volume is permitted thereby providing flow of the biomaterial through the delivery tip and into the introducer cannula.

12. The system of claim 10, further comprising a restraint mechanism coupled to the plunger, wherein the restraint mechanism is configured to restrict axial movement of the plunger within the volume.

13. The system of claim 10, wherein the engagement feature provided at the proximal end of the introducer cannula is a female Luer fitting,
wherein the engagement feature provided at the distal end of the syringe is a male Luer fitting, and
wherein rotational movement between the male and female Luer fittings results in secured coupling between the introducer cannula and the syringe.

14. The system of claim 10, wherein the engagement feature provided at the proximal end of the introducer cannula includes at least one of a recess, a projection, a tapered surface for press fitting with the syringe, a clip, a thread, a bayonet mount,
wherein the engagement feature provided at the distal end of the syringe comprising a corresponding at least one of a projection, a recess, a tapered surface for press fitting with the introducer cannula, a clip, a thread, and a bayonet mount,
wherein coupling movement between the engagement features of the introducer cannula and syringe results in a secured coupling between the introducer cannula and the syringe.

15. The system of claim 10, wherein at least a portion of the delivery tip is coupled to the proximal end of the introducer cannula and extends at least partially into the central channel of the introducer cannula.

16. The system of claim 10, wherein the biomaterial comprises a thermoresponsive polumber comprising a poloxamer.

17. The system of claim 10, wherein the biomaterial comprises a crosslinkable biomaterial,
wherein the syringe further includes:
a second volume separate from the volume containing a second precursor, the volume of the body containing a first precursor, and
where the plunger is sized and configured to separately move within the first and second volumes of the body to convey the first and second precursors through the delivery tip.

18. The system of claim 10, wherein the delivery tip includes an elongated needle defining a first bore and a separate second bore, wherein the first bore is in fluid communication with the first volume of the syringe and the second bore is in fluid communication with the second volume of the syringe, such that the first precursor and the second precursor transmitted through the needle combine as they pass through a distal end of the needle.

19. A method of delivering a stimuli-responsive biomaterial to a treatment site comprising:
inserting an introducer cannula into a body of a patient, wherein a distal end of the introducer cannula is located inside the body of the patient and proximate a treatment site and a proximal end of the introducer cannula is located outside the body of the patient;
inserting a delivery tip of a syringe into the proximal end of the introducer cannula, wherein a stimuli-responsive biomaterial is contained within a body of the syringe;
maintaining the stimuli-responsive biomaterial at a temperature below its liquid-solid phase transition temperature; and
depressing a plunger of the syringe to advance the stimuli-responsive biomaterial in its liquid state through a delivery tip of the syringe and to the treatment site.

20. The method of claim 19, further comprising:
cooling at least one of an interior surface of the body of the syringe and the introducer cannula to a temperature below the liquid-solid phase transition temperature of the stimuli-responsive biomaterial,
wherein the introducer cannula is cooled using a cooled radiofrequency (RF) probe during a cooled RF ablation procedure;
wherein the stimuli-responsive biomaterial is a thermoresponsive polymer comprising a poloxamer.
